(19) United States
(12) Patent Application Publication
(10) Pub. No.: US 2006/0005846 A1
(43) Pub. Date: Jan. 12, 2006

(54) Method for balance enhancement through vestibular, visual, proprioceptive, and cognitive stimulation

(76) Inventors: Wesley W.O. Krueger, San Antonio, TX (US); Lance E. Jackson, San Antonio, TX (US); Jason J. York, San Antonio, TX (US)

Correspondence Address: Michelle L. Evans;Gunn & Lee, P.C., Suite 1500, 700 N. St. Mary's, San Antonio, TX, 78205, US

(21) Appl. No.: 10/885,857

(22) Filed: Jul. 7, 2004

(51) Int. Cl. A61B 19/00

(52) U.S. Cl. 128/898

(57) ABSTRACT

A method for balance enhancement through vestibular, visual, proprioceptive, and cognitive stimulation is provided. This method incorporates head movements in three planes and provides a series of programs for enhancing the vestibular ocular reflex (VOR), dynamic visual acuity, visual pursuit, and associated cognition using these head movements. These programs incorporate (1) head movement with stable image identification, (2) head movement with stable image identification with distractions, (3) head movement with stable image identification and varied body position, (4) data/image identification with varied target frequency, (5) data/image identification with varied screen position, (6) data/image identification with varied target position, and (7) task, memory, and cognitive processing requirements.

### BACKGROUND OF THE INVENTION

1. Field of the Invention

The present invention relates to a method for balance enhancement through vestibular, visual, proprioceptive, and cognitive stimulation.

2. Background Information

The visual, proprioceptive, and vestibular systems are key sensory organ systems responsible for maintaining balance. The visual system receives sensory input through the eyes to determine body position in space and with movement. The vestibular system receives sensory input through the inner ears. The inner ear is sensitive to gravity and detects both linear and angular movements. The proprioceptive system provides information about the relative position of body segments to one another and about the position of the body in space.

When these three systems are functioning properly, balance problems do not typically exist. In addition, these three systems are interdependent on each other which permits balance to be maintained if one of these three primary systems fails. Three resultant mechanisms created by the visual, proprioceptive, and vestibular systems include the oculomotor system, vestibular ocular reflex (VOR), and the vestibular spinal reflex (VSR).

The oculomotor system keeps images centered on the fovea, which is the area of high visual acuity. Dynamic visual acuity is the ability of an individual to quickly fixate and re-fixate on different and moving targets. The three components of this oculomotor system controlled by the central nervous system include: saccades, smooth pursuit, and optokinetics. The saccadic system is responsible for rapidly directing the fovea to a target of interest in visual space. This system creates a conjugate movement of the eyes, a saccade, that brings the fovea on target within a fraction of a second. Saccades are tested by having a patient keep his or her head still while moving only his or her eyes from target to target (typically, the target will appear middle, then left, middle, then right, etc.). The smooth pursuit system is concerned with keeping the fovea on a moving target once that target has been located. Smooth pursuit is tested by having a patient keep his or her head still while smoothly following a moving target with his or her eyes. The optokinetic system detects motion using peripheral vision. The optokinetic system is tested by having a patient keep his or her head still while trying to focus on targets that move rapidly across the patient's field of vision, disappearing on one side and reappearing on the other. Visual pursuit initializes the patient's eye movement or combined eye and head movement to follow a moving object.

A second interdependent system is the VOR. The VOR is a reflex eye movement that stabilizes images on the retina during head movement by producing eye movement in the direction equal and opposite to head movement. If the position of the head is altered, this reflex system keeps the eye looking in the same direction as it did before the movement. Head movement can range from 0 Hz (still) to 14 Hz. The VOR elicits eye movements in response to head movements in all directions, including horizontal, vertical, and diagonal head movements. When head motions are above 2 Hz (two back and forth motions in one second), the VOR is essential to helping maintain balance, because when head motions reach that speed, the smooth pursuit system, the saccadic system, and the optokinetic system cannot effectively function at that speed, and the VOR takes over.

The VOR is measured in the dark to distinguish eye movements driven by vestibular stimuli from eye movements driven by visual stimuli. The performance of the VOR can be measured by the gain, which is defined as the amplitude ratio between eye and head velocities. If a person's VOR gain is poorly calibrated, then head movements result in image motion on the retina, causing blurred vision. Under such conditions, motor learning adjusts the gain of the VOR to produce more accurate eye motion. Such adjustments are needed throughout life, as neurons and muscles develop, weaken, and die or when a new pair of eyeglasses change the magnification of the visual field.

In the laboratory, motor learning for the VOR is induced by pairing image motion with head motion. Depending on the relative direction of head motion and image motion, the gain of the VOR can be adaptively increased or decreased. An increase in VOR gain is induced by image motion in the direction opposite that of the head (gain up stimulus) and a decrease in VOR gain is induced by image motion in the same direction as the head (gain down stimulus).¹
¹There are currently three primary medical diagnostic tools used to objectively evaluate the VOR. These include (1) the Micromedical Technologies System 2000 Rotational Chair, (2) the Micromedical Technologies VORTEQ Active Head Rotation, and (3) the Western Research Systems Vestibular Autorotation Test (VAT®). With Micromedical Technologies System 2000 Rotational Vestibular Chair, the Vestibular Ocular Reflex (VOR) can be measured from 0.01 to 1.28 Hz at velocities up to 300 degrees/second. With the System 2000 chair, the clinician is able to perform VOR, Visual Vestibulo Ocular Reflex (VVOR), Visual Fixation (VFX), Optokinetic After Nystagmus (OKAN), Velocity Step, and Subjective Visual Vertical tests in addition to the standard oculomotor test battery. Micromedical Technologies VORTEQ® Active Head Rotation was designed to provide information about the Vestibular Ocular Reflex (VOR) in patients with dizziness or balance problems. It offers researchers and clinicians a simple and inexpensive method to evaluate VOR gain, phase and symmetry. Specialized software allows the user to conduct the following tests at a velocity range of ±500 degrees/second: (1) Vestibular ocular reflex (VOR), (2) Visual enhanced VOR (VVOR), and (3) Visual Suppression of VOR (VSVOR). Vestibular Autorotation Test (VAT®) was developed as a method of accurately evaluating t(VOR). Unlike the rotational chair, which can test at head speeds only up to 1 Hz, VAT® tests at head speeds of 2-6 Hz, the upper range of head motion speeds people use in their normal, everyday life. VAT® is now in wide use at leading clinics and universities around the globe. With the VAT®, the patient looks at a target and moves his/her head back and forth and then up and down in order to test the VOR.

While the VOR compensates for head movement by using input from the vestibular apparatus to maintain a stable visual image, the optokinetic reflex (OKR) works to maintain a stable image using visual input. The OKR system uses as input visual information coming from the entire retina (not just the fovea) to detect if an image slip is occurring. A slippage is manifest as an optical flow field moving across the retina. A common example of an OKR experience is a person peering out a large window on a moving train, watching the scenery pass by (Furness, 1981). If slippage is occurring, a corrective eye movement is generated to compensate for it by moving the eye with equal gain in the direction of the optical flow. Therefore, while both reflexes serve the same purpose and both can be considered involuntary, the VOR uses vestibular input to generate compensation commands while the OKR uses visual input to do the same. OKR can produce optokinetic nystagmus that appears similar to vestibular induced nystagmus, but while vestibular stimulation (e.g. head rotation) is required for vestibular nystagmus to occur, only a large field-of-view moving image is required to produce optokinetic nystagmus. The reason there are two separate systems to perform essentially the same image stabilizing task is that they both work in synergistic fashion to maximize the eye compensation response to any head movement.

First, consider the fact that even under conditions of enhanced VOR, the gain of the resulting VOR averages approximately 0.95, not the required 1.0 to keep the eyes exactly stabilized in space. Yet the same VOR measured in the light does have a gain equal to 1.0 at most natural frequencies. Therefore, visual information provided by the lighted conditions allows for the correction of VOR residual error through some visual tracking mechanism (Peli, 1995). This visual tracking mechanism is in fact the OKR.

The VOR is a very fast reflex that serves to compensate eye movements effectively for head movements at frequencies in the range of 1-7 Hz, especially if the head movement is voluntary (allowing for efference copy). However, the VOR is less accurate at lower frequencies, especially those lower than 0.1 Hz, where the gain drops significantly and a phase lead appears. The OKR has the opposite performance characteristics. It has longer latency (due to the fact that it uses visual input) than VOR, but at low frequencies (i.e. less than 0.1 Hz), it has near unity gain and no phase difference. From 0.1 Hz to approximately 1 Hz, the OKR begins to lose gain and develop a phase lag due to higher latencies (Peterka, Black & Schoenhoff, 1987). At higher frequencies it cannot effectively compensate due to its relatively long latency and low gain compared to the VOR. Therefore, the combination of the two mechanisms allow for maximal image stabilization all the way from the lowest frequencies (governed mostly by the OKR) to the highest frequencies (governed mostly by the VOR).

There is another aspect of the VOR/OKR combination that contributes to improved performance over either system alone. This aspect is a timing issue: time of onset and time of offset. Earlier it was mentioned that the VOR has a very short latency (onset time) while the OKR has a longer latency. The VOR then allows for a faster reaction time even at lower frequencies. But the VOR will eventually decay during constant, zero-acceleration rotation due to the elasticity of the cupula. Although effectively extended through central processes, the time constant of pure VOR related nystagmus in humans is approximately 25 seconds. The OKR, however, has a long latency but no time constant, as it's response does not decay with repeated stimulation of the retina by an optical flow. Therefore, as the VOR decays, the OKR is building up, creating a continual, seamless stabilization of most images on the retina.

The third interdependent system is the vestibular spinal reflex. The vestibular spinal reflex adjusts posture for rapid changes in position. It helps the maintenance of balance with rapid head movement.

At least two of the three balance-related sensory organ systems (vestibular, visual, and proprioceptive) are necessary to maintain balance, albeit with some difficulty if one of the three is dysfunctional. However, even though the interdependence of the systems may lead to balance compensation when there is a loss of at least one system, other brain functions may suffer as a result. In particular, cognitive difficulties can be caused by disturbances in the balance mechanisms. These difficulties are felt to be a result of suppression of the reticular activating system. Since the areas of the brain that usually carry out thought and memory functions now must focus on balance, the brain sacrifices some of its cognitive function. This leads to a change in mental abilities of the individual.

When an individual appears to be suffering from a balance disorder, the individual can be tested to determine which of the three systems exhibits abnormalities. Numerous tests are available to assess the function of these three systems. The present invention does not concern a diagnostic test, but rather concerns: 1) the enhancement of the appropriate system to permit the individual to regain proper balance, 2) the enhancement of cognitive function for the individual suffering from cognitive deficiency as a result of conscious balance maintenance, and 3) the present invention permits supranormal enhancement of these same systems where no balance disorder exists, as in the case for enhancement of athletic and vocational abilities.

There are currently no known inventions that provide simultaneous enhancement for the vestibular, visual, proprioceptive, and cognitive systems as does the present invention. Such an enhancement methodology can be used in treating balance disorders, athletic/vocational enhancement, and the like. The present invention is useful to (1) persons who have experienced a vestibular insult, such as those caused by infection, concussive injury, vascular disease, surgical complications, Meniere's disease or the like; (2) persons experiencing chronic imbalance, such as, but not limited to, stroke victims, people with systemic illnesses, and the elderly; (3) athletes and others who have experienced head injuries, especially those who have experienced cerebral or labyrinthine (inner ear) concussions; (4) athletes and professionals (e.g. pilots) who wish to enhance their athletic/vocational performance; (5) physician offices that perform vestibular rehabilitation and athletic/vocational enhancement; (6) rehabilitation centers that perform vestibular rehabilitation and athletic/vocational enhancement; (7) athletic programs, ranging from professional sports to high school athletic programs; and (8) fitness centers, sports training centers, athletic performance centers, and vocational performance centers.

### SUMMARY OF THE INVENTION

It is an object of the present invention to provide a novel method for balance enhancement through vestibular stimulation.

Another object of the present invention is to provide a novel method for balance enhancement through visual stimulation.

An additional object of the present invention is to provide a novel method for balance enhancement through proprioceptive stimulation.

It is another object of the present invention to provide a novel method for balance enhancement through cognitive stimulation.

Still another object of the present invention is to provide a novel method to help a person improve his or her balance by challenging, exercising, enhancing, and/or retraining the vestibular ocular reflex used during activities in daily living, routine exercise, and high level athletic/vocational activities.

Yet an additional object of the present invention is to provide a novel method to help a person improve his or her balance by challenging, exercising, enhancing, and/or retraining dynamic visual acuity (fixation/re-fixation)used during activities in daily living, routine exercise, and high level athletic/vocational activities.

Another object of the present invention is to provide a novel method to help a person improve his or her balance by challenging, exercising, enhancing, and/or retraining visual pursuit used during activities in daily living, routine exercise, and high level athletic/vocational activities.

An additional object of the present invention is to provide a novel method to help a person improve his or her balance by challenging, exercising, enhancing, and/or retraining cognitive skills used during activities in daily living, routine exercise, and high level athletic/vocational activities.

In satisfaction of these objectives, the present invention provides a method that incorporates head movements in three planes and provides a series of systematic programs for enhancing the vestibular ocular reflex (VOR), dynamic visual acuity, and visual pursuit using these head movements, with or without cognitive tasks. These programs incorporate (1) head movement with stable image identification, (2) head movement with stable image identification with distractions, (3) head movement with stable image identification and varied body position, (4) data/image identification with varied target frequency, (5) data/image identification with varied screen position, (6) data/image identification with varied target position, and (7) task, memory, and cognitive processing requirements.

### BRIEF DESCRIPTION OF THE DRAWINGS

FIG. 1 is a flow-chart of pre-enhancement protocol.

FIG. 2 is a flow-chart of the general enhancement program.

FIG. 3a is a detailed flow-chart of the horizontal head movement program.

FIG. 3b is a detailed flow-chart of the vertical head movement program.

FIG. 3c is a detailed flow-chart of the diagonal head movement program.

### DETAILED DESCRIPTION OF THE PREFERRED EMBODIMENT

FIG. 1 is a flow-chart of pre-enhancement protocol. The first step in the pre-enhancement protocol is to identify a user in need of balance enhancement, i.e. enhancement of the vestibular ocular reflex (VOR), dynamic visual acuity, visual pursuit, and associated cognition at 104. Next, the enhancement professional will identify the enhancement needed by the user at 106. Once the necessary enhancement is determined, the enhancement professional will identify the program beginning/level of performance for the user at 108. The program beginning/level of performance depends on several characteristics of the user including age, medical condition, specific abnormality, athletic ability, the specific sport or position played where athletic enhancement is involved, the specific vocational skills to improve when professional enhancement is involved, and the goals of the user. Once the program beginning/level of performance has been determined, the professional can proceed to the chosen enhancement program at 110.

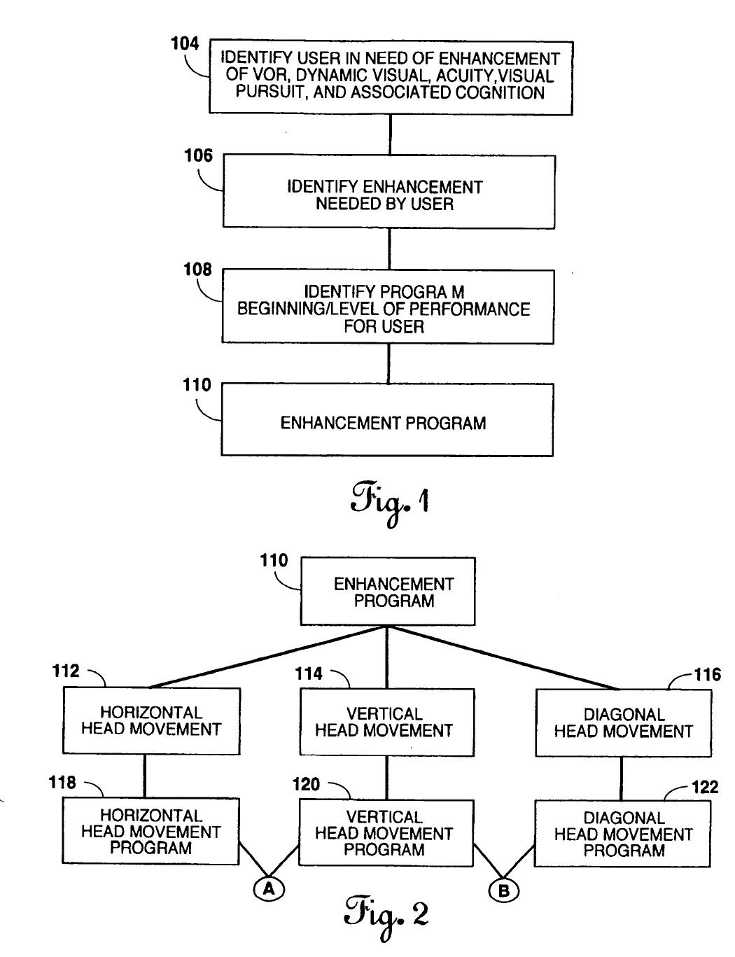

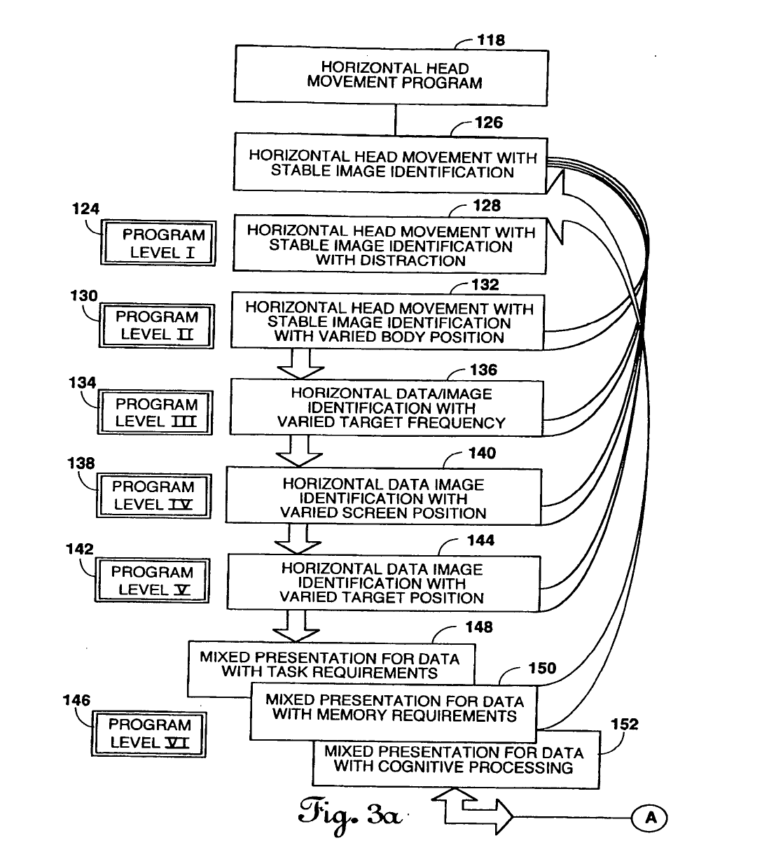

In FIG. 2, a flow-chart of the general enhancement program 110 is provided. The enhancement program chosen depends on the type of head movement involved. When horizontal head movement 112 is involved, the horizontal head movement program 118 will be used. For vertical head movement 114, the vertical head movement program 120 will be used. And when diagonal head movement 116 is involved, the diagonal head movement program 122 will be used. For each of the programs 118, 120, and 122, the frequency of head movement can be fixed or variable. The user may begin and switch between the direction of head movement in either the horizontal, vertical or diagonal plan at any point throughout the process as indicated by the (A) and (B) pathways.

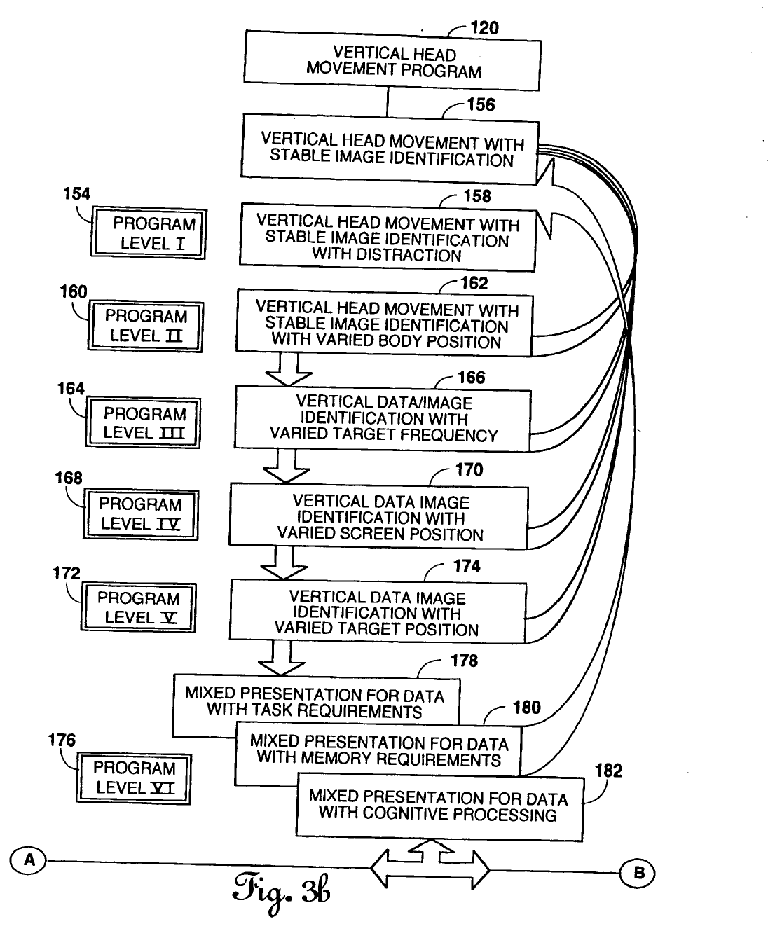

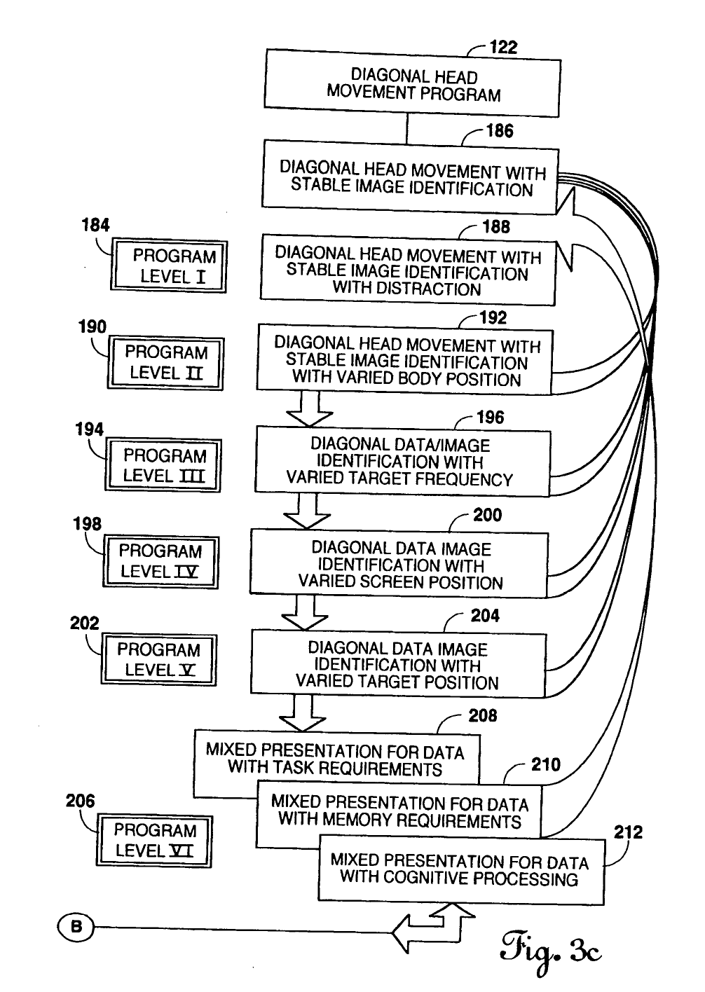

FIG. 3a is a detailed flow-chart of the horizontal head movement program 118. The lowest level of the horizontal head movement program 118 begins with Program Level I at 124. A user may begin at Program Level I or a higher Program Level depending on his or her program beginning/level of performance determined at 108. Program Level I involves horizontal head movement with stable image identification at 126 and/or horizontal head movement with stable image identification with distraction at 128. Image identification involves identification of shapes, numbers, letters, words, combinations of numbers, combination of words, tasks, and cognitive tasks. Distraction includes (1) showing similar shapes on the screen with the same color, (2) showing similar shapes on the screen with different color, (3) showing random shapes on the screen with the same color, (4) showing random shapes on the screen with varying color, (5) playing crowd or vocational-associated noise, and (6) having sentences or words yelled by an “opposing team”, an adversary, placing the participant in another auditory stressful situation while an athlete or professional tries to process cognitive tasks.

To accomplish Program Level I at 124, a computer program is provided which flashes images on a screen for identification by the user. These flashes of images provide the necessary stimulation for the user. During execution of this program, the user is expected to move his or her head from side to side to identify the images. When distractions are introduced in the program, the user is expected to continue horizontal head movements to identify the images on the screen.

As mentioned, a user may begin at Program Level I or a higher Program Level depending on his or her program beginning/level of performance determined at 108. Program Level II at 130 involves horizontal head movement with stable image identification with varied body position at 132. The next step of Program Level II involves combining challenges of varied body position with horizontal head movement with stable image identification with distraction at 128. Again, image identification involves identification of shapes, numbers, letters, words, combinations of numbers, combination of words, tasks, and cognitive tasks. Distraction includes (1) showing similar shapes on the screen with the same color, (2) showing similar shapes on the screen with different color, (3) showing random shapes on the screen with the same color, (4) showing random shapes on the screen with varying color, (5) playing crowd or vocational-associated noise, and (6) having sentences or words yelled by an “opposing team”, an adversary, or placing the participant in another auditory stressful situation while an athlete or professional tries to process cognitive tasks.

Body position includes sitting, standing, riding a stationary bike, walking on a treadmill, running on a treadmill, laying on a foam pad, balancing on a SportKat®, Balance Master®, force plate sensing unit, standing on one leg, jumping on a trampoline, balancing on a wobble board, sport specific body positions, and vocational or other activity specific body positions. Sport specific body positions can include, but are not limited to, jumping, bounding, hurdling, running, walking, standing, crouching, bending, dodging, kicking, dancing, dropping, pulling, stretching, hopping, jogging, hitting, punching, pushing, grabbing, lunging, pressing, squatting, extending arms, extending legs, tossing, throwing, swinging, rotating, twisting, arching, piking, tucking, vaulting, rebounding, swinging, tumbling, resisting, tackling, serving, lifting, and carrying. Vocational specific body positions include, but are not limited to, those body positions specified by the United States Department of Labor as applicable to a given vocation.

To accomplish Program Level II at 130, a computer program is provided which flashes images on a screen for identification by the user. These flashes of images provide the necessary stimulation for the user. During execution of this program, the user will have varying body positions, but is still expected to move his or her head from side to side to identify the images. Next, a computer program is provided which flashes images on a screen for identification by the user. During this program, the user is expected to move his or her head from side to side to identify the images. When distractions are introduced in the program, the user is expected to continue horizontal head movements to identify the images on the screen.

After Program Level II at 130, the enhancement proceeds to Program Level III at 134. Program Level III can also be a starting program depending on the program beginning/level of performance determined at 108. Program Level III at 134 involves horizontal data/image identification with varied target frequency where the user will view images at varying target frequency ranging from 0.01-4.00 seconds (frame time), at 136. This step when completed is directly followed by combining challenges of stable image identification at 126 and/or horizontal head movement with stable image identification with distraction at 128. Again, image identification involves identification of shapes, numbers, letters, words, combinations of numbers, combination of words, tasks, and cognitive tasks. Distraction includes (1) showing similar shapes on the screen with the same color, (2) showing similar shapes on the screen with different color, (3) showing random shapes on the screen with the same color, (4) showing random shapes on the screen with varying color, (5) playing crowd or vocational-associated noise, and (6) having sentences or words yelled by an “opposing team”, an adversary, placing the participant in another auditory stressful situation while an athlete or professional tries to process cognitive tasks.

The next step of Program Level III at 134 involves horizontal head movement with stable image identification with varied body position at 132. Body position includes sitting, standing, riding a stationary bike, walking on a treadmill, running on a treadmill, laying on a foam pad, balancing on a sportkat®, Balance Master®, force plate sensing unit, standing on one leg, jumping on a trampoline, balancing on a wobble board, sport specific body positions, and vocational or other activity specific body positions. Sport specific body positions can include, but are not limited to, jumping, bounding, hurdling, running, walking, standing, crouching, bending, dodging, kicking, dancing, dropping, pulling, stretching, hopping, jogging, hitting, punching, pushing, grabbing, lunging, pressing, squatting, extending arms, extending legs, tossing, throwing, swinging, rotating, twisting, arching, piking, tucking, vaulting, rebounding, swinging, tumbling, resisting, tackling, serving, lifting, and carrying. Vocational specific body positions include, but are not limited to, those body positions specified by the United States Department of Labor as applicable to a given vocation.

To accomplish Program Level III at 134, a computer program is provided which flashes images on a screen for identification by the user. These flashes of images provide the necessary stimulation for the user. During execution of this program, the user will view images at varying target frequency/speed is 0.01-4.00 seconds (frame time), but is still expected to move his or her head from side to side to identify the images. This is followed by a computer program which flashes images on a screen for identification by the user. During this session, the user is expected to move his or her head from side to side to identify the images. When distractions are introduced in the program, the user is expected to continue horizontal head movements to identify the images on the screen. Next, a computer program is provided which flashes images on a screen for identification by the user. During this program, the user will have varying body positions, but is still expected to move his or her head from side to side to identify the images.

After Program Level III at 134, the enhancement proceeds to Program Level IV at 138. Program Level IV can also be a starting program depending on the program beginning/level of performance determined at 108. Program Level IV at 138 involves horizontal data/image identification with varied screen position at 140. Screen position includes positioning of the image on screens at variable locations: (1) one forward screen, (2) two forward screens with varying depths, (3) three forward screens with varying depths, (4) one screen to the side, (5) two screens to the side with varying depths, (6) three screens to the side with varying depths, (7) one forward screen and one side screen, (8) two forward screens and one side screen, (9) one forward screen and two side screens, (10) semicircular screen(s), or (11) domed overhead screen(s).

This step is followed by horizontal head movement with stable image identification at 126 and/or horizontal head movement with stable image identification with distraction at 128. Again, image identification involves identification of shapes, numbers, letters, words, combinations of numbers, combination of words, tasks, and cognitive tasks. Distraction includes (1) showing similar shapes on the screen with the same color, (2) showing similar shapes on the screen with different color, (3) showing random shapes on the screen with the same color, (4) showing random shapes on the screen with varying color, (5) playing crowd or vocational-associated noise, and (6) having sentences or words yelled by an “opposing team”, an adversary, placing the participant in another auditory stressful situation while an athlete or professional tries to process cognitive tasks.

The next step after step 126 involves horizontal head movement with stable image identification with varied body position at 132. Body position includes sitting, standing, riding a stationary bike, walking on a treadmill, running on a treadmill, laying on a foam pad, balancing on a sportkat®, Balance Master®, force plate sensing unit, standing on one leg, jumping on a trampoline, balancing on a wobble board, sport specific body positions, and vocational or other activity specific body positions. Sport specific body positions can include, but are not limited to, jumping, bounding, hurdling, running, walking, standing, crouching, bending, dodging, kicking, dancing, dropping, pulling, stretching, hopping, jogging, hitting, punching, pushing, grabbing, lunging, pressing, squatting, extending arms, extending legs, tossing, throwing, swinging, rotating, twisting, arching, piking, tucking, vaulting, rebounding, swinging, tumbling, resisting, tackling, serving, lifting, and carrying. Vocational specific body positions include, but are not limited to, those body positions specified by the United States Department of Labor as applicable to a given vocation.

This step 132 is followed by horizontal data/image identification with varied target frequency at 136. The variable target frequency/speed for step 136 is 0.01-4.00 second (frame time).

To accomplish Program Level IV at 138, a computer program is provided which flashes images on a screen for identification by the user. These flashes of images provide the necessary stimulation for the user. During execution of the program, the user will view images which generally appear in a horizontal line, but occasionally change screen position. This is followed by a computer program which flashes images on a screen for identification by the user. During this program, the user is expected to move his or her head from side to side to identify the images. When distractions are introduced in the program, the user is expected to continue horizontal head movements to identify the images on the screen. After this test, a computer program is provided which requires the user to vary his or her body position while viewing the images, but is still expected to move his or her head from side to side to identify the images. Next, a computer program is provided which requires the user to view images at varying target frequencies, but is still expected to move his or her head from side to side to identify the images.

After Program Level IV at 138, the enhancement proceeds to Program Level V at 142. Program Level V can also be a starting program depending on the program beginning/level of performance determined at 108. Program Level V at 142 involves horizontal data/image identification with varied target position at step 144. Target position includes positioning the image (1) straight ahead in center of the screen, (2) straight ahead in center of screen with head moving, (3) alternating targets in horizontal plane, (4) alternating targets in vertical plane, (5) alternating targets in diagonal plane, (6) alternating targets in random sports or vocational settings, (7) alternating targets in specific sports or vocational settings, or (8) target tracking in horizontal, vertical, diagonal or random planes.

This step 144 is followed by horizontal head movement with stable image identification at 126 and/or horizontal head movement with stable image identification with distraction at 128. Again, image identification involves identification of shapes, numbers, letters, words, combinations of numbers, combination of words, tasks, and cognitive tasks. Distraction includes (1) showing similar shapes on the screen with the same color, (2) showing similar shapes on the screen with different color, (3) showing random shapes on the screen with the same color, (4) showing random shapes on the screen with varying color, (5) playing crowd or vocational-associated noise, and (6) having sentences or words yelled by an “opposing team”, an adversary, placing the participant in another auditory stressful situation while an athlete or professional tries to process cognitive tasks.

The next step involves horizontal head movement with stable image identification with varied body position at 132. Body position includes sitting, standing, riding a stationary bike, walking on a treadmill, running on a treadmill, laying on a foam pad, balancing on a sportkat®, Balance Master®, force plate sensing unit, standing on one leg, jumping on a trampoline, balancing on a wobble board, sport specific body positions, and vocational or other activity specific body positions. Sport specific body positions can include, but are not limited to, jumping, bounding, hurdling, running, walking, standing, crouching, bending, dodging, kicking, dancing, dropping, pulling, stretching, hopping, jogging, hitting, punching, pushing, grabbing, lunging, pressing, squatting, extending arms, extending legs, tossing, throwing, swinging, rotating, twisting, arching, piking, tucking, vaulting, rebounding, swinging, tumbling, resisting, tackling, serving, lifting, and carrying. Vocational specific body positions include, but are not limited to, those body positions specified by the United States Department of Labor as applicable to a given vocation.

This step 132 is followed by horizontal data/image identification with varied target frequency at 136. The variable target frequency/speed for step 136 is 0.01-4.00 second (frame time). After step 136, the program proceeds to horizontal data/image identification with varied screen position at 140. Screen position includes positioning of the image on screens at (1) one forward screen, (2) two forward screens with varying depths, (3) three forward screens with varying depths, (4) one screen to the side, (5) two screens to the side with varying depths, (6) three screens to the side with varying depths, (7) one forward screen and one side screen, (8) two forward screens and one side screen, (9) one forward screen and two side screens, (10) semicircular screen(s), or (11) domed overhead screen(s).

To accomplish Program Level V at 142, a computer program is provided which flashes images on a screen for identification by the user. These flashes of images provide the necessary stimulation for the user. During execution of the program, the user will view images which change target position in all three planes. This is followed by a computer program which flashes images on a screen for identification by the user. During execution of this program, the user is expected to move his or her head from side to side to identify the images. When distractions are introduced in the program, the user is expected to continue horizontal head movements to identify the images on the screen. After this test, a computer program is provided which requires the user to vary his or her body position while viewing the images, but the user is still expected to move his or her head from side to side to identify the images. Next, a computer program is provided which requires the user to view images at varying frequencies, but the user is still expected to move his or her head from side to side to identify the images. Next, a computer program is provided which flashes images on a screen for identification by the user. During execution of the program, the user will view images which generally appear in a horizontal line, but occasionally change screen position.

After Program Level V at 142, the enhancement proceeds to Program Level VI at 146. Program Level VI can also be a starting program depending on the program beginning/level of performance determined at 108. At level 146, the user is asked to incorporate certain task 148, memory 150 and cognitive 152 processing requirements while performing the requirements of Program Levels I through V. A typical task requirement at step 148 would be for a user to orally identify words or numbers flashed on a screen. With regard to the memory requirement at step 150, the user views a series of images flashed in different positions on the screen and is then asked to remember and recite those images. With regard to the cognitive processing step at 152, the user is presented with a series of boxes with words, numbers or objects. The user is then required to remember the images which were flashed and provide a knowledge based response to a series of questions. For example, the user may be required to add a series of numbers and give the total.

Program Level VI 146 is followed by horizontal head movement with stable image identification at 126 and/or horizontal head movement with stable image identification with distraction at 128. Again, image identification involves identification of shapes, numbers, letters, words, combinations of numbers, combination of words, tasks, and cognitive tasks. Distraction includes (1) showing similar shapes on the screen with the same color, (2) showing similar shapes on the screen with different color, (3) showing random shapes on the screen with the same color, (4) showing random shapes on the screen with varying color, (5) playing crowd or vocational-associated noise, and (6) having sentences or words yelled by an “opposing team”, an adversary, placing the participant in another auditory stressful situation while an athlete or professional tries to process cognitive tasks.

The next step involves horizontal head movement with stable image identification with varied body position at 132. Body position includes sitting, standing, riding a stationary bike, walking on a treadmill, running on a treadmill, laying on a foam pad, balancing on a sportkat®, Balance Master®, force plate sensing unit, standing on one leg, jumping on a trampoline, balancing on a wobble board, sport specific body positions, and vocational or other activity specific body positions. Sport specific body positions can include, but are not limited to, jumping, bounding, hurdling, running, walking, standing, crouching, bending, dodging, kicking, dancing, dropping, pulling, stretching, hopping, jogging, hitting, punching, pushing, grabbing, lunging, pressing, squatting, extending arms, extending legs, tossing, throwing, swinging, rotating, twisting, arching, piking, tucking, vaulting, rebounding, swinging, tumbling, resisting, tackling, serving, lifting, and carrying. Vocational specific body positions include, but are not limited to, those body positions specified by the United States Department of Labor as applicable to a given vocation.

This step 132 is followed by horizontal data/image identification with target varied frequency at 136. The variable target frequency/speed for step 136 is 0.01-4.00 second (frame time). After step 136, the program proceeds to horizontal data/image identification with varied screen position at 140. Screen position includes positioning of the image on screens at (1) one forward screen, (2) two forward screens with varying depths, (3) three forward screens with varying depths, (4) one screen to the side, (5) two screens to the side with varying depths, (6) three screens to the side with varying depths, (7) one forward screen and one side screen, (8) two forward screens and one side screen, (9) one forward screen and two side screens, (10) semicircular screen(s), or (11) domed overhead screen(s).

Step 144 involves horizontal data/image identification with varied target position. Target position includes positioning the image (1) straight ahead in center of the screen, (2) straight ahead in center of screen with head moving, (3) alternating targets in horizontal plane, (4) alternating targets in vertical plane, (5) alternating targets in diagonal plane, (6) alternating targets at random sports or vocational screen, (7) alternating targets in specific sports or vocational settings, or (8) target tracking in horizontal, vertical, diagonal or random planes.

To accomplish Program Level VI at 146, a computer program is provided which flashes images on a screen for identification by the user. These flashes of images provide the necessary stimulation for the user. During execution of the program, the user will view images which stimulate task requirements, memory requirements, and/or cognitive requirement by the user. During execution of this program, the user is expected to move his or her head from side to side to identify the images. When distractions are introduced in the program, the user is expected to continue horizontal head movements to identify the images on the screen.

After this test, a computer program is provided which requires the user to vary his or her body position while viewing the images, but the user is still expected to move his or her head from side to side to identify the images. Next, a computer program is provided which requires the user to view images at varying frequencies, but the user is still expected to move his or her head from side to side to identify the images. Next, a computer program is provided which flashes images on a screen for identification by the user. During execution of the program, the user will view images which generally appear in a horizontal line, but occasionally change screen position.

FIG. 3b is a detailed flow-chart of the vertical head movement program 120. The lowest level of the vertical head movement program 120 begins with Program Level I at 154. A user may begin at Program Level I or a higher Program Level depending on his or her program beginning/level of performance determined at 108. Program Level I involves vertical head movement with stable image identification at 156 and/or vertical head movement with stable image identification with distraction at 158. Image identification involves identification of shapes, numbers, letters, words, combinations of numbers, combination of words, tasks, and cognitive tasks. Distraction includes (1) showing similar shapes on the screen with the same color, (2) showing similar shapes on the screen with different color, (3) showing random shapes on the screen with the same color, (4) showing random shapes on the screen with varying color, (5) playing crowd or vocational-associated noise, and (6) having sentences or words yelled by an “opposing team”, an adversary, placing the participant in another auditory stressful situation while an athlete or professional tries to process cognitive tasks.

To accomplish Program Level I at 154, a computer program is provided which flashes images on a screen for identification by the user. These flashes of images provide the necessary stimulation for the user. During execution of this program, the user is expected to move his or her head from side to side to identify the images. When distractions are introduced in the program, the user is expected to continue vertical head movements to identify the images on the screen.

As mentioned, a user may begin at Program Level I or a higher Program Level depending on his or her program beginning/level of performance determined at 108. Program Level II at 160 involves vertical head movement with stable image identification with varied body position at 162. The next step of Program Level II involves combining challenges of varied body position with vertical head movement with stable image identification with distraction at 158. Again, image identification involves identification of shapes, numbers, letters, words, combinations of numbers, combination of words, tasks, and cognitive tasks. Distraction includes (1) showing similar shapes on the screen with the same color, (2) showing similar shapes on the screen with different color, (3) showing random shapes on the screen with the same color, (4) showing random shapes on the screen with varying color, (5) playing crowd or vocational-associated noise, and (6) having sentences or words yelled by an “opposing team”, an adversary, placing the participant in another auditory stressful situation while an athlete or professional tries to process cognitive tasks.

Body position includes sitting, standing, riding a stationary bike, walking on a treadmill, running on a treadmill, laying on a foam pad, balancing on a sportkat®, Balance Master®, force plate sensing unit, standing on one leg, jumping on a trampoline, balancing on a wobble board, sport specific body positions, and vocational or other activity specific body positions. Sport specific body positions can include, but are not limited to, jumping, bounding, hurdling, running, walking, standing, crouching, bending, dodging, kicking, dancing, dropping, pulling, stretching, hopping, jogging, hitting, punching, pushing, grabbing, lunging, pressing, squatting, extending arms, extending legs, tossing, throwing,'swinging, rotating, twisting, arching, piking, tucking, vaulting, rebounding, swinging, tumbling, resisting, tackling, serving, lifting, and carrying. Vocational specific body positions include, but are not limited to, those body positions specified by the United States Department of Labor as applicable to a given vocation.

To accomplish Program Level II at 160, a computer program is provided which flashes images on a screen for identification by the user. These flashes of images provide the necessary stimulation for the user. During execution of this program, the user will have varying body positions, but is still expected to move his or her head from side to side to identify the images. Next, a computer program is provided which flashes images on a screen for identification by the user. During this program, the user is expected to move his or her head from side to side to identify the images. When distractions are introduced in the program, the user is expected to continue vertical head movements to identify the images on the screen.

After Program Level II at 160, the enhancement proceeds to Program Level III at 164. Program Level III can also be a starting program depending on the program beginning/level of performance determined at 108. Program Level III at 164 involves vertical data/image identification with varied target frequency were the user will view images at varying target frequency/speed ranging from 0.01-4.00 seconds (frame time), at 166. This step when completed is directly followed by combining challenges of stable image identification at 156 and/or vertical head movement with stable image identification with distraction at 158. Again, image identification involves identification of shapes, numbers, letters, words, combinations of numbers, combination of words, tasks, and cognitive tasks. Distraction includes (1) showing similar shapes on the screen with the same color, (2) showing similar shapes on the screen with different color, (3) showing random shapes on the screen with the same color, (4) showing random shapes on the screen with varying color, (5) playing crowd or vocational-associated noise, and (6) having sentences or words yelled by an “opposing team”, an adversary, placing the participant in another auditory stressful situation while an athlete or professional tries to process cognitive tasks.

The next step of Program Level III at 164 involves vertical head movement with stable image identification with varied body position at 162. Body position includes sitting, standing, riding a stationary bike, walking on a treadmill, running on a treadmill, laying on a foam pad, balancing on a sportkat®, Balance Master®, force plate sensing unit, standing on one leg, jumping on a trampoline, balancing on a wobble board, sport specific body positions, and vocational or other activity specific body positions. Sport specific body positions can include, but are not limited to, jumping, bounding, hurdling, running, walking, standing, crouching, bending, dodging, kicking, dancing, dropping, pulling, stretching, hopping, jogging, hitting, punching, pushing, grabbing, lunging, pressing, squatting, extending arms, extending legs, tossing, throwing, swinging, rotating, twisting, arching, piking, tucking, vaulting, rebounding, swinging, tumbling, resisting, tackling, serving, lifting, and carrying. Vocational specific body positions include, but are not limited to, those body positions specified by the United States Department of Labor as applicable to a given vocation.

To accomplish Program Level III at 164, a computer program is provided which flashes images on a screen for identification by the user. These flashes of images provide the necessary stimulation for the user. During execution of this program, the user will view images at varying target frequency/speed is 0.01-4.00 seconds (frame time), but is still expected to move his or her head from side to side to identify the images. This is followed by a computer program which flashes images on a screen for identification by the user. During this session, the user is expected to move his or her head from side to side to identify the images. When distractions are introduced in the program, the user is expected to continue vertical head movements to identify the images on the screen. Next, a computer program is provided which flashes images on a screen for identification by the user. During this program, the user will have varying body positions, but is still expected to move his or her head from side to side to identify the images.

After Program Level III at 164, the enhancement proceeds to Program Level IV at 168. Program Level IV can also be a starting program depending on the program beginning/level of performance determined at 108. Program Level IV at 168 involves vertical data/image identification with varied screen position at 170. Screen position includes positioning of the image on screens at variable locations: (1) one forward screen, (2) two forward screens with varying depths, (3) three forward screens with varying depths, (4) one screen to the side, (5) two screens to the side with varying depths, (6) three screens to the side with varying depths, (7) one forward screen and one side screen, (8) two forward screens and one side screen, (9) one forward screen and two side screens, (10) semicircular screen(s), or (11) domed overhead screen(s).

This step is followed by vertical head movement with stable image identification at 156 and/or vertical head movement with stable image identification with distraction at 158. Again, image identification involves identification of shapes, numbers, letters, words, combinations of numbers, combination of words, tasks, and cognitive tasks. Distraction includes (1) showing similar shapes on the screen with the same color, (2) showing similar shapes on the screen with different color, (3) showing random shapes on the screen with the same color, (4) showing random shapes on the screen with varying color, (5) playing crowd or vocational-associated noise, and (6) having sentences or words yelled by an “opposing team”, an adversary, placing the participant in another auditory stressful situation while an athlete or professional tries to process cognitive tasks.

The next step after step 156 involves vertical head movement with stable image identification with varied body position at 162. Body position includes sitting, standing, riding a stationary bike, walking on a treadmill, running on a treadmill, laying on a foam pad, balancing on a sportkat®, Balance Master®, force plate sensing unit, standing on one leg, jumping on a trampoline, balancing on a wobble board, sport specific body positions, and vocational or other activity specific body positions. Sport specific body positions can include, but are not limited to, jumping, bounding, hurdling, running, walking, standing, crouching, bending, dodging, kicking, dancing, dropping, pulling, stretching, hopping, jogging, hitting, punching, pushing, grabbing, lunging, pressing, squatting, extending arms, extending legs, tossing, throwing, swinging, rotating, twisting, arching, piking, tucking, vaulting, rebounding, swinging, tumbling, resisting, tackling, serving, lifting, and carrying. Vocational specific body positions include, but are not limited to, those body positions specified by the United States Department of Labor as applicable to a given vocation.

This step 162 is followed by vertical data/image identification with varied target frequency at 166. The variable target frequency/speed for step 166 is 0.01-4.00 second (frame time).

To accomplish Program Level IV at 168, a computer program is provided which flashes images on a screen for identification by the user. These flashes of images provide the necessary stimulation for the user. During execution of the program, the user will view images which generally appear in a vertical line, but occasionally change screen position. This is followed by a computer program which flashes images on a screen for identification by the user. During this program, the user is expected to move his or her head from side to side to identify the images. When distractions are introduced in the program, the user is expected to continue vertical head movements to identify the images on the screen. After this test, a computer program is provided which requires the user to vary his or her body position while viewing the images, but is still expected to move his or her head from side to side to identify the images. Next, a computer program is provided which requires the user to view images at varying target frequencies, but is still expected to move his or her head from side to side to identify the images.

After Program Level IV at 168, the enhancement proceeds to Program Level V at 172. Program Level V can also be a starting program depending on the program beginning/level of performance determined at 108. Program Level V at 172 involves vertical data/image identification with varied target position at step 174. Target position includes positioning the image (1) straight ahead in center of the screen, (2) straight ahead in center of screen with head moving, (3) alternating targets in vertical plane, (4) alternating targets in vertical plane, (5) alternating targets in diagonal plane, (6) alternating targets in random sports or vocational settings, (7) alternating targets in specific sports or vocational settings, or (8) target tracking in vertical, vertical, diagonal or random planes.

This step 174 is followed by vertical head movement with stable image identification at 156 and/or vertical head movement with stable image identification with distraction at 158. Again, image identification involves identification of shapes, numbers, letters, words, combinations of numbers, combination of words, tasks, and cognitive tasks. Distraction includes (1) showing similar shapes on the screen with the same color, (2) showing similar shapes on the screen with different color, (3) showing random shapes on the screen with the same color, (4) showing random shapes on the screen with varying color, (5) playing crowd or vocational-associated noise, and (6) having sentences or words yelled by an “opposing team”, an adversary, placing the participant in another auditory stressful situation while an athlete or professional tries to process cognitive tasks.

The next step involves vertical head movement with stable image identification with varied body position at 162. Body position includes sitting, standing, riding a stationary bike, walking on a treadmill, running on a treadmill, laying on a foam pad, balancing on a sportkat®, Balance Master®, force plate sensing unit, standing on one leg, jumping on a trampoline, balancing on a wobble board, sport specific body positions, and vocational or other activity specific body positions. Sport specific body positions can include, but are not limited to, jumping, bounding, hurdling, running, walking, standing, crouching, bending, dodging, kicking, dancing, dropping, pulling, stretching, hopping, jogging, hitting, punching, pushing, grabbing, lunging, pressing, squatting, extending arms, extending legs, tossing, throwing, swinging, rotating, twisting, arching, piking, tucking, vaulting, rebounding, swinging, tumbling, resisting, tackling, serving, lifting, and carrying. Vocational specific body positions include, but are not limited to, those body positions specified by the United States Department of Labor as applicable to a given vocation.

This step 162 is followed by vertical data/image identification with varied target frequency at 166. The variable target frequency/speed for step 166 is 0.01-4.00 second (frame time). After step 166, the program proceeds to vertical data/image identification with varied screen position at 170. Screen position includes positioning of the image on screens at (1) one forward screen, (2) two forward screens with varying depths, (3) three forward screens with varying depths, (4) one screen to the side, (5) two screens to the side with varying depths, (6) three screens to the side with varying depths, (7) one forward screen and one side screen, (8) two forward screens and one side screen, (9) one forward screen and two side screens, (10) semicircular screen(s), or (11) domed overhead screen(s).

To accomplish Program Level V at 172, a computer program is provided which flashes images on a screen for identification by the user. These flashes of images provide the necessary stimulation for the user. During execution of the program, the user will view images which change target position in all three planes. This is followed by a computer program which flashes images on a screen for identification by the user. During execution of this program, the user is expected to move his or her head from side to side to identify the images. When distractions are introduced in the program, the user is expected to continue vertical head movements to identify the images on the screen. After this test, a computer program is provided which requires the user to vary his or her body position while viewing the images, but the user is still expected to move his or her head from side to side to identify the images. Next, a computer program is provided which requires the user to view images at varying frequencies, but the user is still expected to move his or her head from side to side to identify the images. Next, a computer program is provided which flashes images on a screen for identification by the user. During execution of the program, the user will view images which generally appear in a vertical line, but occasionally change screen position.

After Program Level V at 172, the enhancement proceeds to Program Level VI at 176. Program Level VI can also be a starting program depending on the program beginning/level of performance determined at 108. At level 176, the user is asked to incorporate certain task 178, memory 180 and cognitive 182 processing requirements while performing the requirements of Program Levels I through V. A typical task requirement at step 178 would be for a user to orally identify words or numbers flashed on a screen. With regard to the memory requirement at step 180, the user views a series of images flashed in different positions on the screen and is then asked to remember and recite those images. With regard to the cognitive processing step at 182, the user is presented with a series of boxes with words, numbers or objects. The user is then required to remember the images which were flashed and provide a knowledge based response to a series of questions. For example, the user may be required to add a series of numbers and give the total.

Program Level VI 176 is followed by vertical head movement with stable image identification at 156 and/or vertical head movement with stable image identification with distraction at 158. Again, image identification involves identification of shapes, numbers, letters, words, combinations of numbers, combination of words, tasks, and cognitive tasks. Distraction includes (1) showing similar shapes on the screen with the same color, (2) showing similar shapes on the screen with different color, (3) showing random shapes on the screen with the same color, (4) showing random shapes on the screen with varying color, (5) playing crowd or vocational-associated noise, and (6) having sentences or words yelled by an “opposing team”, an adversary, placing the participant in another auditory stressful situation while an athlete or professional tries to process cognitive tasks.

The next step involves vertical head movement with stable image identification with varied body position at 162. Body position includes sitting, standing, riding a stationary bike, walking on a treadmill, running on a treadmill, laying on a foam pad, balancing on a sportkat®, Balance Master®, force plate sensing unit, standing on one leg, jumping on a trampoline, balancing on a wobble board, sport specific body positions, and vocational or other activity specific body positions. Sport specific body positions can include, but are not limited to, jumping, bounding, hurdling, running, walking, standing, crouching, bending, dodging, kicking, dancing, dropping, pulling, stretching, hopping, jogging, hitting, punching, pushing, grabbing, lunging, pressing, squatting, extending arms, extending legs, tossing, throwing, swinging, rotating, twisting, arching, piking, tucking, vaulting, rebounding, swinging, tumbling, resisting, tackling, serving, lifting, and carrying. Vocational specific body positions include, but are not limited to, those body positions specified by the United States Department of Labor as applicable to a given vocation.

This step 162 is followed by vertical data/image identification with target varied frequency at 166. The variable target frequency/speed for step 166 is 0.01-4.00 second (frame time). After step 166, the program proceeds to vertical data/image identification with varied screen position at 170. Screen position includes positioning of the image on screens at (1) one forward screen, (2) two forward screens with varying depths, (3) three forward screens with varying depths, (4) one screen to the side, (5) two screens to the side with varying depths, (6) three screens to the side with varying depths, (7) one forward screen and one side screen, (8) two forward screens and one side screen, (9) one forward screen and two side screens, (10) semicircular screen(s), or (11) domed overhead screen(s).

Step 174 involves vertical data/image identification with varied target position. Target position includes positioning the image (1) straight ahead in center of the screen, (2) straight ahead in center of screen with head moving, (3) alternating targets in vertical plane, (4) alternating targets in vertical plane, (5) alternating targets in diagonal plane, (6) alternating targets at random sports or vocational screen, (7) alternating targets in specific sports or vocational settings, or (8) target tracking in vertical, vertical, diagonal or random planes.

To accomplish Program Level VI at 176, a computer program is provided which flashes images on a screen for identification by the user. These flashes of images provide the necessary stimulation for the user. During execution of the program, the user will view images which stimulate task requirements, memory requirements, and/or cognitive requirement by the user. During execution of this program, the user is expected to move his or her head from side to side to identify the images. When distractions are introduced in the program, the user is expected to continue vertical head movements to identify the images on the screen.

After this test, a computer program is provided which requires the user to vary his or her body position while viewing the images, but the user is still expected to move his or her head from side to side to identify the images. Next, a computer program is provided which requires the user to view images at varying frequencies, but the user is still expected to move his or her head from side to side to identify the images. Next, a computer program is provided which flashes images on a screen for identification by the user. During execution of the program, the user will view images which generally appear in a vertical line, but occasionally change screen position.

FIG. 3c is a detailed flow-chart of the diagonal head movement program 122. The lowest level of the diagonal head movement program 122 begins with Program Level I at 184. A user may begin at Program Level I or a higher Program Level depending on his or her program beginning/level of performance determined at 108. Program Level I involves diagonal head movement with stable image identification at 186 and/or diagonal head movement with stable image identification with distraction at 188. Image identification involves identification of shapes, numbers, letters, words, combinations of numbers, combination of words, tasks, and cognitive tasks. Distraction includes (1) showing similar shapes on the screen with the same color, (2) showing similar shapes on the screen with different color, (3) showing random shapes on the screen with the same color, (4) showing random shapes on the screen with varying color, (5) playing crowd or vocational-associated noise, and (6) having sentences or words yelled by an “opposing team”, an adversary, placing the participant in another auditory stressful situation while an athlete or professional tries to process cognitive tasks.

To accomplish Program Level I at 184, a computer program is provided which flashes images on a screen for identification by the user. These flashes of images provide the necessary stimulation for the user. During execution of this program, the user is expected to move his or her head from side to side to identify the images. When distractions are introduced in the program, the user is expected to continue diagonal head movements to identify the images on the screen.

As mentioned, a user may begin at Program Level I or a higher Program Level depending on his or her program beginning/level of performance determined at 108. Program Level II at 190 involves diagonal head movement with stable image identification with varied body position at 192. The next step of Program Level II involves combining challenges of varied body position with diagonal head movement with stable image identification with distraction at 188. Again, image identification involves identification of shapes, numbers, letters, words, combinations of numbers, combination of words, tasks, and cognitive tasks. Distraction includes (1) showing similar shapes on the screen with the same color, (2) showing similar shapes on the screen with different color, (3) showing random shapes on the screen with the same color, (4) showing random shapes on the screen with varying color, (5) playing crowd or vocational-associated noise, and (6) having sentences or words yelled by an “opposing team”, an adversary, placing the participant in another auditory stressful situation while an athlete or professional tries to process cognitive tasks.

Body position includes sitting, standing, riding a stationary bike, walking on a treadmill, running on a treadmill, laying on a foam pad, balancing on a sportkat®, Balance Master®, force plate sensing unit, standing on one leg, jumping on a trampoline, balancing on a wobble board, sport specific body positions, and vocational or other activity specific body positions. Sport specific body positions can include, but are not limited to, jumping, bounding, hurdling, running, walking, standing, crouching, bending, dodging, kicking, dancing, dropping, pulling, stretching, hopping, jogging, hitting, punching, pushing, grabbing, lunging, pressing, squatting, extending arms, extending legs, tossing, throwing, swinging, rotating, twisting, arching, piking, tucking, vaulting, rebounding, swinging, tumbling, resisting, tackling, serving, lifting, and carrying. Vocational specific body positions include, but are not limited to, those body positions specified by the United States Department of Labor as applicable to a given vocation.

To accomplish Program Level II at 190, a computer program is provided which flashes images on a screen for identification by the user. These flashes of images provide the necessary stimulation for the user. During execution of this program, the user will have varying body positions, but is still expected to move his or her head from side to side to identify the images. Next, a computer program is provided which flashes images on a screen for identification by the user. During this program, the user is expected to move his or her head from side to side to identify the images. When distractions are introduced in the program, the user is expected to continue diagonal head movements to identify the images on the screen.

After Program Level II at 190, the enhancement proceeds to Program Level III at 194. Program Level III can also be a starting program depending on the program beginning/level of performance determined at 108. Program Level III at 194 involves diagonal data/image identification with varied target frequency were the user will view images at varying target frequency/speed ranging from 0.01-4.00 seconds (frame time), at 196. This step when completed is directly followed by combining challenges of stable image identification at 186 and/or diagonal head movement with stable image identification with distraction at 188. Again, image identification involves identification of shapes, numbers, letters, words, combinations of numbers, combination of words, tasks, and cognitive tasks. Distraction includes (1) showing similar shapes on the screen with the same color, (2) showing similar shapes on the screen with different color, (3) showing random shapes on the screen with the same color, (4) showing random shapes on the screen with varying color, (5) playing crowd or vocational-associated noise, and (6) having sentences or words yelled by an “opposing team”, an adversary, placing the participant in another auditory stressful situation while an athlete or professional tries to process cognitive tasks.

The next step of Program Level III at 194 involves diagonal head movement with stable image identification with varied body position at 192. Body position includes sitting, standing, riding a stationary bike, walking on a treadmill, running on a treadmill, laying on a foam pad, balancing on a sportkat®, Balance Master®, force plate sensing unit, standing on one leg, jumping on a trampoline, balancing on a wobble board, sport specific body positions, and vocational or other activity specific body positions. Sport specific body positions can include, but are not limited to, jumping, bounding, hurdling, running, walking, standing, crouching, bending, dodging, kicking, dancing, dropping, pulling, stretching, hopping, jogging, hitting, punching, pushing, grabbing, lunging, pressing, squatting, extending arms, extending legs, tossing, throwing, swinging, rotating, twisting, arching, piking, tucking, vaulting, rebounding, swinging, tumbling, resisting, tackling, serving, lifting, and carrying. Vocational specific body positions include, but are not limited to, those body positions specified by the United States Department of Labor as applicable to a given vocation.

To accomplish Program Level III at 194, a computer program is provided which flashes images on a screen for identification by the user. These flashes of images provide the necessary stimulation for the user. During execution of this program, the user will view images at varying target frequency/speed is 0.01-4.00 seconds (frame time), but is still expected to move his or her head from side to side to identify the images. This is followed by a computer program which flashes images on a screen for identification by the user. During this session, the user is expected to move his or her head from side to side to identify the images. When distractions are introduced in the program, the user is expected to continue diagonal head movements to identify the images on the screen. Next, a computer program is provided which flashes images on a screen for identification by the user. During this program, the user will have varying body positions, but is still expected to move his or her head from side to side to identify the images.

After Program Level III at 194, the enhancement proceeds to Program Level IV at 198. Program Level IV can also be a starting program depending on the program beginning/level of performance determined at 108. Program Level IV at 198 involves diagonal data/image identification with varied screen position at 200. Screen position includes positioning of the image on screens at variable locations: (1) one forward screen, (2) two forward screens with varying depths, (3) three forward screens with varying depths, (4) one screen to the side, (5) two screens to the side with varying depths, (6) three screens to the side with varying depths, (7) one forward screen and one side screen, (8) two forward screens and one side screen, (9) one forward screen and two side screens, (10) semicircular screen(s), or (11) domed overhead screen(s).

This step is followed by diagonal head movement with stable image identification at 186 and/or diagonal head movement with stable image identification with distraction at 188. Again, image identification involves identification of shapes, numbers, letters, words, combinations of numbers, combination of words, tasks, and cognitive tasks. Distraction includes (1) showing similar shapes on the screen with the same color, (2) showing similar shapes on the screen with different color, (3) showing random shapes on the screen with the same color, (4) showing random shapes on the screen with varying color, (5) playing crowd or vocational-associated noise, and (6) having sentences or words yelled by an “opposing team”, an adversary, placing the participant in another auditory stressful situation while an athlete or professional tries to process cognitive tasks.

The next step after step 186 involves diagonal head movement with stable image identification with varied body position at 192. Body position includes sitting, standing, riding a stationary bike, walking on a treadmill, running on a treadmill, laying on a foam pad, balancing on a sportkat®, Balance Master®, force plate sensing unit, standing on one leg, jumping on a trampoline, balancing on a wobble board, sport specific body positions, and vocational or other activity specific body positions. Sport specific body positions can include, but are not limited to, jumping, bounding, hurdling, running, walking, standing, crouching, bending, dodging, kicking, dancing, dropping, pulling, stretching, hopping, jogging, hitting, punching, pushing, grabbing, lunging, pressing, squatting, extending arms, extending legs, tossing, throwing, swinging, rotating, twisting, arching, piking, tucking, vaulting, rebounding, swinging, tumbling, resisting, tackling, serving, lifting, and carrying. Vocational specific body positions include, but are not limited to, those body positions specified by the United States Department of Labor as applicable to a given vocation.

This step 192 is followed by diagonal data/image identification with varied target frequency at 196. The variable target frequency/speed for step 196 is 0.01-4.00 second (frame time).

To accomplish Program Level IV at 198, a computer program is provided which flashes images on a screen for identification by the user. These flashes of images provide the necessary stimulation for the user. During execution of the program, the user will view images which generally appear in a diagonal line, but occasionally change screen position. This is followed by a computer program which flashes images on a screen for identification by the user. During this program, the user is expected to move his or her head from side to side to identify the images. When distractions are introduced in the program, the user is expected to continue diagonal head movements to identify the images on the screen. After this test, a computer program is provided which requires the user to vary his or her body position while viewing the images, but is still expected to move his or her head from side to side to identify the images. Next, a computer program is provided which requires the user to view images at varying target frequencies, but is still expected to move his or her head from side to side to identify the images.

After Program Level IV at 198, the enhancement proceeds to Program Level V at 202. Program Level V can also be a starting program depending on the program beginning/level of performance determined at 108. Program Level V at 202 involves diagonal data/image identification with varied target position at step 204. Target position includes positioning the image (1) straight ahead in center of the screen, (2) straight ahead in center of screen with head moving, (3) alternating targets in diagonal plane, (4) alternating targets in vertical plane, (5) alternating targets in diagonal plane, (6) alternating targets in random sports or vocational settings, (7) alternating targets in specific sports or vocational settings, or (8) target tracking in diagonal, vertical, diagonal or random planes.

This step 204 is followed by diagonal head movement with stable image identification at 186 and/or diagonal head movement with stable image identification with distraction at 188. Again, image identification involves identification of shapes, numbers, letters, words, combinations of numbers, combination of words, tasks, and cognitive tasks. Distraction includes (1) showing similar shapes on the screen with the same color, (2) showing similar shapes on the screen with different color, (3) showing random shapes on the screen with the same color, (4) showing random shapes on the screen with varying color, (5) playing crowd or vocational-associated noise, and (6) having sentences or words yelled by an “opposing team”, an adversary, placing the participant in another auditory stressful situation while an athlete or professional tries to process cognitive tasks.

The next step involves diagonal head movement with stable image identification with varied body position at 192. Body position includes sitting, standing, riding a stationary bike, walking on a treadmill, running on a treadmill, laying on a foam pad, balancing on a sportkat®, Balance Master®, force plate sensing unit, standing on one leg, jumping on a trampoline, balancing on a wobble board, sport specific body positions, and vocational or other activity specific body positions. Sport specific body positions can include, but are not limited to, jumping, bounding, hurdling, running, walking, standing, crouching, bending, dodging, kicking, dancing, dropping, pulling, stretching, hopping, jogging, hitting, punching, pushing, grabbing, lunging, pressing, squatting, extending arms, extending legs, tossing, throwing, swinging, rotating, twisting, arching, piking, tucking, vaulting, rebounding, swinging, tumbling, resisting, tackling, serving, lifting, and carrying. Vocational specific body positions include, but are not limited to, those body positions specified by the United States Department of Labor as applicable to a given vocation.

This step 192 is followed by diagonal data/image identification with varied target frequency at 196. The variable target frequency/speed for step 196 is 0.01-4.00 second (frame time). After step 196, the program proceeds to diagonal data/image identification with varied screen position at 200. Screen position includes positioning of the image on screens at (1) one forward screen, (2) two forward screens with varying depths, (3) three forward screens with varying depths, (4) one screen to the side, (5) two screens to the side with varying depths, (6) three screens to the side with varying depths, (7) one forward screen and one side screen, (8) two forward screens and one side screen, (9) one forward screen and two side screens, (10) semicircular screen(s), or (11) domed overhead screen(s).

To accomplish Program Level V at 202, a computer program is provided which flashes images on a screen for identification by the user. These flashes of images provide the necessary stimulation for the user. During execution of the program, the user will view images which change target position in all three planes. This is followed by a computer program which flashes images on a screen for identification by the user. During execution of this program, the user is expected to move his or her head from side to side to identify the images. When distractions are introduced in the program, the user is expected to continue diagonal head movements to identify the images on the screen. After this test, a computer program is provided which requires the user to vary his or her body position while viewing the images, but the user is still expected to move his or her head from side to side to identify the images. Next, a computer program is provided which requires the user to view images at varying frequencies, but the user is still expected to move his or her head from side to side to identify the images. Next, a computer program is provided which flashes images on a screen for identification by the user. During execution of the program, the user will view images which generally appear in a diagonal line, but occasionally change screen position.

After Program Level V at 202, the enhancement proceeds to Program Level VI at 206. Program Level VI can also be a starting program depending on the program beginning/level of performance determined at 108. At level 206, the user is asked to incorporate certain task 208, memory 210 and cognitive 212 processing requirements while performing the requirements of Program Levels I through V. A typical task requirement at step 208 would be for a user to orally identify words or numbers flashed on a screen. With regard to the memory requirement at step 210, the user views a series of images flashed in different positions on the screen and is then asked to remember and recite those images. With regard to the cognitive processing step at 212, the user is presented with a series of boxes with words, numbers or objects. The user is then required to remember the images which were flashed and provide a knowledge based response to a series of questions. For example, the user may be required to add a series of numbers and give the total.

Program Level VI 206 is followed by diagonal head movement with stable image identification at 186 and/or diagonal head movement with stable image identification with distraction at 188. Again, image identification involves identification of shapes, numbers, letters, words, combinations of numbers, combination of words, tasks, and cognitive tasks. Distraction includes (1) showing similar shapes on the screen with the same color, (2) showing similar shapes on the screen with different color, (3) showing random shapes on the screen with the same color, (4) showing random shapes on the screen with varying color, (5) playing crowd or vocational-associated noise, and (6) having sentences or words yelled by an “opposing team”, an adversary, placing the participant in another auditory stressful situation while an athlete or professional tries to process cognitive tasks.

The next step involves diagonal head movement with stable image identification with varied body position at 192. Body position includes sitting, standing, riding a stationary bike, walking on a treadmill, running on a treadmill, laying on a foam pad, balancing on a sportkat®, Balance Master®, force plate sensing unit, standing on one leg, jumping on a trampoline, balancing on a wobble board, sport specific body positions, and vocational or other activity specific body positions. Sport specific body positions can include, but are not limited to, jumping, bounding, hurdling, running, walking, standing, crouching, bending, dodging, kicking, dancing, dropping, pulling, stretching, hopping, jogging, hitting, punching, pushing, grabbing, lunging, pressing, squatting, extending arms, extending legs, tossing, throwing, swinging, rotating, twisting, arching, piking, tucking, vaulting, rebounding, swinging, tumbling, resisting, tackling, serving, lifting, and carrying. Vocational specific body positions include, but are not limited to, those body positions specified by the United States Department of Labor as applicable to a given vocation.

This step 192 is followed by diagonal data/image identification with target varied frequency at 196. The variable target frequency/speed for step 196 is 0.01-4.00 second (frame time). After step 196, the program proceeds to diagonal data/image identification with varied screen position at 200. Screen position includes positioning of the image on screens at (1) one forward screen, (2) two forward screens with varying depths, (3) three forward screens with varying depths, (4) one screen to the side, (5) two screens to the side with varying depths, (6) three screens to the side with varying depths, (7) one forward screen and one side screen, (8) two forward screens and one side screen, (9) one forward screen and two side screens, (10) semicircular screen(s), or (11) domed overhead screen(s).

Step 204 involves diagonal data/image identification with varied target position. Target position includes positioning the image (1) straight ahead in center of the screen, (2) straight ahead in center of screen with head moving, (3) alternating targets in diagonal plane, (4) alternating targets in vertical plane, (5) alternating targets in diagonal plane, (6) alternating targets at random sports or vocational screen, (7) alternating targets in specific sports or vocational settings, or (8) target tracking in diagonal, vertical, diagonal or random planes.

To accomplish Program Level VI at 206, a computer program is provided which flashes images on a screen for identification by the user. These flashes of images provide the necessary stimulation for the user. During execution of the program, the user will view images which stimulate task requirements, memory requirements, and/or cognitive requirement by the user. During execution of this program, the user is expected to move his or her head from side to side to identify the images. When distractions are introduced in the program, the user is expected to continue diagonal head movements to identify the images on the screen.

After this test, a computer program is provided which requires the user to vary his or her body position while viewing the images, but the user is still expected to move his or her head from side to side to identify the images. Next, a computer program is provided which requires the user to view images at varying frequencies, but the user is still expected to move his or her head from side to side to identify the images. Next, a computer program is provided which flashes images on a screen for identification by the user. During execution of the program, the user will view images which generally appear in a diagonal line, but occasionally change screen position.

For each of the computer programs detailed herein, the images are preferably displayed on a curved screen for full enhancement benefits as a curved screen provides greater depth for peripheral vision. However, the computer programs can be modified for display on a flat screen. The order and manner in which the images are flashed is computer controlled.

Although the invention has been described with reference to specific embodiments, this description is not meant to be construed in a limited sense. Various modifications of the disclosed embodiments, as well as alternative embodiments of the inventions will become apparent to persons skilled in the art upon the reference to the description of the invention. All such similar substitutes and modifications apparent to those skilled in the art are deemed to be within the spirit, scope and concept of the invention as defined by the appended claims.

We claim:

1. A method for balance enhancement comprising the steps of:
identifying a user in need of balance enhancement;
selecting an enhancement program based on characteristics of the user and head movement necessary for said balance enhancement, wherein said enhancement program incorporates head movement with stable image identification; and executing said enhancement program.

2. The method for balance enhancement of claim 1 wherein said enhancement program incorporates head movement with stable image identification and distraction.

3. The method for balance enhancement of claim 2 wherein said enhancement program incorporates head movement with stable image identification and varied body position.

4. The method for balance enhancement of claim 3 wherein said enhancement program incorporates data/image identification with varied target frequency.

5. The method for balance enhancement of claim 4 wherein said enhancement program incorporates data/image identification with varied screen position.

6. The method for balance enhancement of claim 5 wherein said enhancement program incorporates data/image identification with varied target position.

7. The method for balance enhancement of claim 6 wherein said enhancement program incorporates task processing.

8. The method for balance enhancement of claim 7 wherein said enhancement program incorporates memory processing.

9. The method for balance enhancement of claim 8 wherein said enhancement program incorporates cognitive processing.

10. The method for balance enhancement of claim 1 wherein said characteristics of said user comprise age, medical condition, abnormality, sport athletic ability, vocation and user's goals.

11. The method for balance enhancement of claim 10 wherein said head movement is selected from the group consisting of horizontal, vertical, and diagonal head movements.

12. The method for balance enhancement of claim 1 wherein said image identification is selected from the group consisting of identification of shapes, numbers, letters, words, combinations of numbers, combinations of words, tasks and cognitive tasks.

13. The method for balance enhancement of claim 2 wherein said distraction is selected from the group consisting of showing similar shapes on a screen, said shapes having the same color; showing similar shapes on a screen, said shapes having different color; showing random shapes on a screen, said random shapes having the same color; showing random shapes on a screen, said random shapes having different color; playing crowd or vocational-associated noise; and yelling sentences or words.

14. The method for balance enhancement of claim 3 wherein said body position includes sitting, standing, riding a stationary bike, walking on a treadmill, running on a treadmill, laying on a foam pad, balancing on a sportkat®, Balance Master®, force plate sensing unit, standing on one leg, jumping on a trampoline, balancing on a wobble board, sport specific body positions, and vocational or other activity specific body positions. Sport specific body positions can include, but are not limited to, jumping, bounding, hurdling, running, walking, standing, crouching, bending, dodging, kicking, dancing, dropping, pulling, stretching, hopping, jogging, hitting, punching, pushing, grabbing, lunging, pressing, squatting, extending arms, extending legs, tossing, throwing, swinging, rotating, twisting, arching, piking, tucking, vaulting, rebounding, swinging, tumbling, resisting, tackling, serving, lifting, and carrying. Vocational specific body positions include, but are not limited to, those body positions specified by the United States Department of Labor as applicable to a given vocation.

15. The method for balance enhancement of claim 4 wherein said target frequency is based on a 0.01-4.00 second (frame time).

16. The method for balance enhancement of claim 5 wherein said screen position is selected from the group consisting of one forward screen, two forward screens with varying depths, three forward screens with varying depths, one screen to the side, two screens to the side with varying depths, three screens to the side with varying depths, one forward screen and one side screen, two forward screens and one side screen, one forward screen and two side screens, at least one semicircular screen, and at least one domed overhead screen.

17. The method for balance enhancement of claim 6 wherein said target position is selected from the group consisting of a position straight ahead in the center of the screen, a position straight ahead in the center of the screen with the head moving, alternating targets in the horizontal plane, alternating targets in the vertical plane, alternating targets in the diagonal plane, alternating targets at random sports or vocational screens, alternating targets in specific sports or vocational settings and target tracking in horizontal, vertical, diagonal, or random planes.
